(19) United States
(12) Patent Application Publication
(10) Pub. No.: US 2011/0172210 A1
(43) Pub. Date: Jul. 14, 2011

(54) METHOD FOR TITRATING CLOZAPINE

(75) Inventors: Seamus MULLIGAN, Dublin (IE); Fintan KEEGAN, Athlone (IE)

(73) Assignee: AZUR PHARMA LIMITED, Dublin (IE)

(21) Appl. No.: 12/987,660

(22) Filed: Jan. 10, 2011

Related U.S. Application Data
(60) Provisional application No. 61/294,692, filed on Jan. 13, 2010.

(51) Int. Cl. A61K 31/551; A61P 25/18

(52) U.S. Cl. 514/220

(57) ABSTRACT

Method for titrating clozapine in treatment of psychotic disorders in a human patient in need of treatment with clozapine including administering once-a-day controlled release clozapine to the human patient titrating to a therapeutic dose of clozapine within a period of less than one week, or administering controlled release clozapine to the patient at an initial dose of at least 37.5 mg and titrating to a therapeutic dose within a period of less than ten days.

### CROSS-REFERENCE TO RELATED APPLICATIONS

This application claims the benefit of U.S. Provisional Application No. 61/294,692, filed Jan. 13, 2010, the disclosure of which is incorporated by reference herein in its entirety.

### BACKGROUND OF THE INVENTION

1. Field of the Invention

The present invention relates to the titration of clozapine, and particularly to titration of clozapine in a manner that permits rapid titration to be obtained. The present invention also relates to controlled release formulations containing clozapine. The present invention also relates to solid oral dosage forms containing clozapine, and the administration of such dosage forms, including titration of clozapine. The present invention also relates to the treatment of psychotic disorders, such as schizophrenia, schizoaffective disorder, and dementia-related psychosis, in mammals, particularly humans, that includes rapid titration to therapeutic levels.

2. Discussion of Background Information

Clozapine is used for the management of severely ill schizophrenic patients who fail to respond adequately to standard drug treatment for schizophrenia. Clozapine is also used for reducing the risk of recurrent suicidal behavior in patients with schizophrenia or schizoaffective disorder who are judged to be at chronic risk for reexperiencing suicidal behavior, based on history and recent clinical state. Clozapine is also used in the treatment of Parkinson related psychosis. Suicidal behavior refers to actions by a patient that put him/herself at risk for death.

Clozapine is the preferred treatment for patients with refractory schizophrenia that have had inadequate or no response to other antipsychotic therapy. See, for example, Y. W. Francis Lam et al., “Branded Versus Generic Clozapine: Bioavailability Comparison and Interchangeability Issues,” J Clin Psychiatry 2001; 62 (suppl. 5), 18-24, which is incorporated by reference herein in its entirety.

Clozapine has the following chemical structure and name:

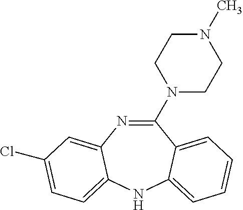

### 8-chloro-11-(4-methyl-1-piperazinyl)-5H-dibenzo[b,e][1,4]diazepine

C₁₈H₁₉ClN₄ having a molecular weight of 326.83

The exact mechanism by which clozapine exerts its antipsychotic effects has not been elucidated. Clozapine is termed an atypical antipsychotic because it lacks extrapyramidal effects (such as tardive dyskinesia) commonly observed with typical antipsychotics, including chlorpromazine and haloperidol. Clozapine has affinity for the serotonin (5-HT2A, 5-HT2C, 5-HT6, and 5-HT7), muscarinic m1, and α 1-adrenergic receptors.

Additionally, clozapine acts as a receptor antagonist at the dopamine D1, D3, and D5 receptors with preference for the D4 receptor and weak affinity for the D2 receptor. It is the low affinity at the D2 receptor that is proposed to provide advantages of clozapine over other neuroleptic compounds. It has also been hypothesized that the D1/D2 antagonist activity may, along with the D4 receptor specificity, be responsible for the decrease in side effects observed.

Another hypothesis to explain the mechanism of clozapine's effect is based on elevated plasma norepinephrine levels. Clozapine is thought to elevate arterial plasma norepinephrine by increasing norepinephrine spillover, with no effect on norepinephrine synthesis or metabolism.

Adverse events (AEs) observed in association with the use of clozapine reference product in clinical trials at an incidence of greater than 5% are central nervous system complaints including drowsiness/sedation, dizziness/vertigo, headache, tremor, and syncope; autonomic nervous system complaints, including salivation, sweating, dry mouth, and visual disturbances; cardiovascular findings, including tachycardia and hypotension; gastrointestinal complaints, including constipation and nausea; and miscellaneous complaints, including fever. Complaints of drowsiness/sedation tend to subside with continued therapy or dose reduction. Salivation may be profuse, especially during sleep, but may be diminished with dose reduction.

Less frequent serious adverse events (SAES) observed in association with the use of clozapine include agranulocytosis, seizures, myocarditis, electrocardiogram (ECG) repolarization changes, hyperglycemia and diabetes mellitus; other adverse cardiovascular and respiratory events including orthostatic hypotension and respiratory and/or cardiac arrest and cardiomyopathy.

In fact, due to risk of serious adverse events, the Prescribing Information for FazaClo® (clozapine, USP) Orally Disintegrating Tablets, SPL for Approved Supplement SLR-013, NDA 021590, August 2008, pages 1-34, which is incorporated by reference herein in its entirety, specifically provides bold-faced, capital letter warnings of agranulocytosis, seizures, myocarditis, other adverse cardiovascular and respiratory effects, and increased mortality in elderly patients with dementia-related psychosis.

Clozapine formulations accordingly include warnings for seizures, myocarditis, and other adverse cardiovascular and respiratory effects. Lowering of the seizure threshold is dose related, Cmax, and slow initial titration of dose is practiced in a desire to decrease the risk of precipitating seizures. Slow titration of dosing may also decrease the risk for seizures and orthostatic hypotension and other adverse cardiovascular side effects, and an anti-cholinergic affect. Clozapine patient insert leaflets specify that titration should be at no more than 25 to 50 mg per day. A patient can take 10 or more days to receive a therapeutic dose, such as a dose of 400 mg or more, and reaching the desired therapeutic dose can take weeks. See, for example, Iqbal et al., “Clozapine: A Clinical Review of Adverse Effects and Management”; Annals of Clinical Psychiatry, Vol. 15, No. 1, pages 33-48, March 2003, which is incorporated by reference herein in its entirety.

For example, for the initial treatment, it is recommended that treatment with CLOZARIL® (clozapine) begin with one-half of a 25 mg tablet (12.5 mg) once or twice daily and then be continued with daily dosage increments of 25-50 mg/day, if well-tolerated, to achieve a target dose of 300-450 mg/day by the end of 2 weeks. Moreover, the package insert indicates that subsequent dosage increments should be made no more than once or twice-weekly, in increments not to exceed 100 mg. Cautious titration and a divided dosage schedule are stated to be necessary to minimize the risks of hypotension, seizure, and sedation, and anti-cholinergic effect. It is noted in the insert that, “In the multicenter study that provides primary support for the effectiveness of CLOZARIL® (clozapine) in patients resistant to standard drug treatment for schizophrenia, patients were titrated during the first 2 weeks up to a maximum dose of 500 mg/day, on a t.i.d. basis, and were then dosed in a total daily dose range of 100-900 mg/day, on a t.i.d. basis thereafter, with clinical response and adverse effects as guides to correct dosing.”

Therapeutic dose adjustment is indicated as daily dosing should continue on a divided basis as an effective and tolerable dose level is sought. While many patients may respond adequately at doses between 300-600 mg/day, it may be necessary to raise the dose to the 600-900 mg/day range to obtain an acceptable response. It is noted that in the multicenter study providing the primary support for the superiority of CLOZARIL® (clozapine) in treatment resistant patients, the mean and median CLOZARIL® (clozapine) doses were both approximately 600 mg/day. Again, it is disclosed that, “Because of the possibility of increased adverse reactions at higher doses, particularly seizures, patients should ordinarily be given adequate time to respond to a given dose level before escalation to a higher dose is contemplated.” It is disclosed that CLOZARIL® (clozapine) can cause EEG changes, including the occurrence of spike and wave complexes. It lowers the seizures threshold in a dose-dependent manner and may induce myoclonic jerks or generalized seizures. These symptoms may be likely to occur with rapid dose increase and in patients with pre-existing epilepsy. In this case, the dose should be reduced and, if necessary, anticonvulsant treatment initiated.

Clozapine is prescribed for refractory patients, i.e., patients who have not responded to other antipsychotic drugs, such as resperidal or zyprexa, but the FDA has approved clozapine for reducing suicidal behaviour. While patients do respond better to clozapine, doctors are still hesitant about using it especially as they have seriously ill patients with a high suicidal tendency. This is because it may take two weeks or more to titrate to a therapeutic level.

Thus, while there is a need to achieve therapeutic effects in patients such as to achieve therapeutic levels of clozapine to address suicidal behavior, the conventional manner of administration of clozapine requires unduly long titration times.

### SUMMARY OF THE INVENTION

This invention provides for rapid titration with clozapine.

This invention provides for rapid titration with clozapine in a once-a-day formulation.

This invention also provides for rapid titration with clozapine in a once-a-day formulation at an initial higher daily dose than 12.5 mg or 25 mg of clozapine.

There is provided herein a method for titrating clozapine in treatment of a psychotic disorder in a human patient in need of treatment with clozapine, comprising administering once-a-day controlled release clozapine to the human patient and titrating to a therapeutic dose within a period of less than one week.

There is also provided herein a method for titrating clozapine in treatment of a psychotic disorder in a human patient in need of treatment with clozapine, comprising administering once-a-day controlled release clozapine to the human patient and titrating to a therapeutic dose of at least 100 mg, at least 200 mg, at least 250 mg or at least 300 mg within a period of less than one week.

The once-a-day controlled release clozapine can be administered at an initial dose of 25 mg.

There is also provided a method for titrating clozapine in treatment of a psychotic disorder in a human patient in need of treatment with clozapine, comprising administering once-a-day controlled release clozapine to the human patient at an initial dose of at least 37.5 mg of clozapine and titrating to a therapeutic dose within a period of less than ten days.

The therapeutic dose can be a daily dose of at least 100 mg of clozapine, at least 200 mg of clozapine, at least 250 mg, at least 300 mg of clozapine, at least 350 mg of clozapine, at least 400 mg of clozapine, at least 450 mg of clozapine, at least 500 mg of clozapine, at least 550 mg of clozapine, at least 600 mg of clozapine, at least 650 mg of clozapine or at least 700 mg of clozapine. A therapeutic dose range can be 100 mg of clozapine to 900 mg of clozapine, or 200 mg of clozapine to 700 mg of clozapine, or 300 mg of clozapine to 600 mg of clozapine, or 300 mg of clozapine to 500 mg of clozapine, or 300 mg of clozapine to 450 mg of clozapine.

The administering can be to obtain a therapeutic dose in one week, a therapeutic dose in less than one week, a therapeutic dose in four days, a therapeutic dose in three days, or a therapeutic dose in two days or in one day.

The once-a-day controlled release clozapine can comprise a dosage form including one component, can comprise a dosage form comprising a single unit, and can comprise a dosage from including two or more components.

The once-a-day controlled release clozapine can comprise a dosage form comprising single unit, such as a dosage form including 37.5 mg of clozapine in a single unit.

The initial dose can comprise 50 mg of clozapine, 100 mg of clozapine, 150 mg of clozapine, 200 mg of clozapine, 250 mg of clozapine, 300 mg of clozapine, or 350 mg of clozapine, or higher. The initial dose can therefore comprise at least 50 mg of clozapine, at least 100 mg of clozapine, at least 150 mg of clozapine, at least 200 mg of clozapine, at least 250 mg of clozapine, at least 300 mg of clozapine, or at least 350 mg of clozapine, or higher.

The titrating can comprise increasing each once-a-day dose following the initial dose by at least 50 mg of clozapine, by at least 100 mg of clozapine, by at least 150 mg of clozapine, by at least 200 mg of clozapine and or by at least 250 mg of clozapine, or higher.

The once-a-day controlled release clozapine can comprise, for a 200 mg daily dose of clozapine, a steady state blood Cₘₐₓ level of clozapine in the range of 200 ng/mL to about 500 ng/mL.

The daily dose of 200 mg of clozapine can obtain steady state blood Cₘᵢₙ of clozapine of 50 to 350 ng/mL.

The AUC can be 4000 to 10000 ng/mL*hr.

The daily dose can be administered in the evening before bedtime.

The daily dose can be administered at bedtime.

The daily dose can be administered in the morning

The daily dose can be administered at any time during the day.

The administering of clozapine can be performed to obtain an improvement in schizophrenia as measured by Positive and Negative Syndrome Scale (PANSS). There can be a decrease in sedation or a delay in the onset of sedation. There is an improvement or stabilization in patient mood.

The administering of clozapine can be performed to minimize central nervous system complaints; autonomic nervous system complaints; cardiovascular findings; gastrointestinal complaints; and/or miscellaneous complaints.

The administering of clozapine can be performed to minimize agranulocytosis, seizures, myocarditis, electrocardiogram (ECG) repolarization changes, hyperglycemia and diabetes mellitus; other adverse cardiovascular and respiratory events and/or anti-cholinergic affect.

The administering of clozapine can be performed to minimize orthostatic hypotension, respiratory and/or cardiac arrest, and cardiomyopathy.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 illustrates a graphical representation of dose concentration over time for exemplary simulations;

FIG. 2 illustrates a graphical representation of dose concentration over time for exemplary simulations;

FIG. 3 illustrates a graphical representation of dose concentration over time for exemplary simulations; and

FIG. 4 illustrates a graphical representation of dose concentration over time for exemplary simulations.

### DETAILED DESCRIPTION OF THE INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description is taken with the drawings making apparent to those skilled in the art how the forms of the present invention may be embodied in practice.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the disclosure of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range. Moreover, the terminology at least includes the stated number, e.g., “at least 50” includes 50.

The pharmacokinetic profiles Cₘₐₓ, Tₘₐₓ, and (AUC) are defined as follows:

Cₘₐₓ (ng/mL): Maximum plasma concentration;

Tₘₐₓ (hr): Time of maximum plasma concentration;

AUC: Area under the plasma concentration time curve which is a measure of drug absorption as defined by (AUCO₀₋ₜ) and (AUC_{inf});

(AUCO₀₋ₜ) (hr*ng/mL): AUC versus time curve from time 0 to the time t of the last quantifiable concentration, calculated by means of the mixed log-linear trapezoidal rule;

(AUC_{inf}) (hr*ng/mL) The AUC value versus time curve from time 0 to infinity, calculated as (AUC_{inf})=(AUC₀₋ₜ) AUCₑₓₜᵣₐ. AUCₑₓₜᵣₐ represents an extrapolated value obtained by Ct/λz, where Ct is the observed concentration at time t at or above LOQ, and λz is the estimated apparent terminal rate constant; and

Cₘᵢₙ is minimum or “trough” plasma concentration (Cₘᵢₙ) of a drug observed after its administration and just prior to the administration of a subsequent dose.

There is provided the achieving of a faster titration regime when administering clozapine. When administering a controlled released formulation of clozapine, there is obtained a concentration of clozapine or desmethylclozapine, in the blood of up to 24 hours that results in effectively being similar with respect to AUC of an immediate clozapine formulation that is administered twice daily. The solubility of clozapine is pH dependent and increases in the stomach but decreases extensively in the gut. An immediate release clozapine will precipitate out in the duodenum and will not be as bioavailable. By controlling the release of clozapine, as compared to immediate release, there will not be this expected precipitation seen in the upper intestine. It has unexpectedly been determined that by practicing the administration schedule of the present invention a rapid titration of clozapine can be achieved, e.g., therapeutic levels of clozapine can be reached in a period of ten days or less, including one week or less than one week, and preferably within 2 to 4, or even 2 to 3 days. This is unexpected as conventional titration schedules can take as long as two weeks or longer. Moreover, it is unexpected that the titration can be more rapidly completed while still avoiding serious side affects that would be expected when performing a rapid titration with clozapine.

Thus, for example, there is provided a method for titrating clozapine in treatment of psychotic disorders in a human patient in need of treatment with at least one of clozapine, comprising administering at least one of once-a-day controlled release clozapine to the patient to obtain titration to a therapeutic dose within a period of less than one week.

There is also provided a faster dose titration regime by starting at a higher clozapine dose, such as an initial clozapine dose of at least 37.5 mg, at least 50 mg, at least 100 mg, at least 150 mg, at least 200 mg, at least 250 mg, at least 300 mg, at least 350 mg, or at least 400 mg, (and including any value inbetween 37.5 mg and 400 mg of clozapine) rather than an initial dose of 12.5 mg, and reaching therapeutic levels using any of these higher initial dose levels within ten days or less, or one week or less than one week, and even as rapidly as in 2 to 4 days, or 2 to 3 days, or even one day. Moreover, additional doses can comprise increasing each dose by one or more of 50 mg of clozapine, 100 mg of clozapine, 150 mg of clozapine, 200 mg of clozapine, 250 mg of clozapine, 300 mg of clozapine, or even higher.

Thus, the embodiments herein, provide for alternative methods of titrating clozapine in treatment of psychotic disorders in a human patient in need of treatment with clozapine by permitting use of an initial dose such as a once-day-controlled release formulation of 25 mg, as disclosed below, and titrating at a fast rate to obtain titration in a period of less than one week, or provide for administering controlled release clozapine to the patient at an initial dose of at least 37.5 mg, or at least 50 mg or greater to obtain titration to a therapeutic dose within a period of less than ten days.

The present invention provides rapid titration of clozapine using controlled release of clozapine, preferably in a once-a-day controlled release formulation. For example, and without wishing to be bound by theory, by having low or no immediate release component, the Cₘₐₓ of the pharmacokinetic profile (PK profile) will be expected to be significantly reduced compared to immediate release twice a day (BID) clozapine products on the market. As noted above, clozapine has warnings for seizures, myocarditis, and other adverse cardiovascular and respiratory effects. Lowering of the seizure threshold is dose related, Cₘₐₓ, and slow initial titration of dose should thereby decrease the risk for precipitating seizures. Slow titration of dosing may also decrease the risk for orthostatic hypotension and other adverse cardiovascular side effects. However, as discussed above, while patients respond better to clozapine, doctors are still hesitant about using it especially as they have seriously ill patients with high suicidal tendency. This is because it may take two weeks to titrate to a therapeutic level. Accordingly, the present invention provides the advantage of shorter titration times, and the ability to rapidly achieve therapeutic levels of clozapine administration as well as to the ability to begin with a higher dose.

Therapeutic level or dose as used herein means that the patient is brought to a level of drug administration that is adequate to achieve a minimum therapeutic effect. Thus, the amount of clozapine administered is sufficient to elicit the required or desired therapeutic response, or in other words, the amount is sufficient to elicit an appreciable effective response to stabilize a patient when administered to the patient. This minimum can be as low as 100 mg of clozapine a day. The dosage level of clozapine can be thereafter be increased to one or more higher therapeutic levels by increasing the dosing with further titration, such as titrating up to about 900 mg of clozapine a day or higher. A typical therapeutic dose is a dose of 300 mg of clozapine a day or higher with a typical range being 300 mg of clozapine a day to 450 mg of clozapine a day.

For example, to reach 100 mg of clozapine per day using twice a day dosing (BID or b.i.d.) will take a minimum of five days. Whereas to reach 100 mg of clozapine a day following titration using a once-daily formulation can be achieved in three days or less. Similarly, for example, to reach 900 mg of clozapine per day using b.i.d. dosing will take a minimum of 28 days; whereas, to reach 900 mg of clozapine a day following titration using a once-daily formulation can be achieved in seven days or less.

Expanding upon the above, for example, a patient not previously taking clozapine can be initially administered immediate release clozapine by first taking a 12.5 mg tablet and increasing the dose by no more than 50 mg daily in divided doses. The period of time to reach a daily dose of 350 mg from a starting dose of 12.5 mg is not less than or typically more than 8 days. In contrast, a once-a-day controlled release formulation can start with a 50 mg capsule or tablet and increase the daily dose by 100 mg or more per day, so as to reach a daily dose of 350 mg in 7, 6, 5, 4 or 3 days. A physician can monitor the period and judge the titration dose according to severity and frequency of adverse events, such as orthostatic hypertension, somnolence, seizures and delirium.

As used herein, “administering” means the patient has received and ingested clozapine given to the patient either by taking the clozapine himself; a medical professional, including but not limited to a doctor or nurse, gives the patient the dose of clozapine; or any person gives the dose of clozapine to the patient. The patient is a mammal, preferably a human.

The clozapine administered according to the present invention can be included in various formulations, and is preferably included in a controlled release formulation, more preferably a once-a-day controlled release formulation. Controlled release refers to the release of an agent such as a drug from a composition or dosage form in which the agent is released according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic or diagnostic response as compared to conventional rapid release dosage forms. Such longer periods of response provide for many inherent benefits that are not achieved with the corresponding short acting, immediate release preparations. For example, in the treatment of chronic pain, controlled release formulations are often highly preferred over conventional short-acting formulations. See, for example, patent documents which disclose controlled release clozapine formulations including U.S. Pat. No. 6,150,424 to Breitenbach et al., U.S. Pat. No. 6,210,712 B1 to Edgren et al., and U.S. Published Patent Application Nos. 2002/0012675 A1 to Jain et al., 2008/0124393 A1 to Jain et al., 2010/0260859 A1 to Ruddy et al. and 2010/0260858 A1 to Ruddy et al. which are incorporated by reference herein in their entireties.

For example, the Jain et al. published patent applications disclose controlled release nanoparticulate compositions providing for the therapeutically effective release of an incorporated drug or other substance in a patient for a time period ranging from about 2 to about 24 hours or longer, and US 2010/0260859 A1 discloses a pharmaceutical dosage form of clozapine having a therapeutic effect for up to 24 hours.

As noted above, controlled release pharmaceutical compositions and dosage forms are designed to improve the delivery profile of agents, such as drugs, medicaments, active agents, diagnostic agents, or any substance to be internally administered to an animal, including mammals, such as humans. A controlled release composition is typically used to improve the effects of administered substances by optimizing the kinetics of delivery, thereby increasing bioavailability, convenience, and patient compliance, as well as minimizing side effects associated with inappropriate immediate release rates such as a high initial release rate and, if undesired, uneven blood or tissue levels.

In contrast, immediate release of a drug comprises the release of the drug without modification of its release rate. Immediate release of drugs typically results in a high initial blood level of the drug with a need for frequent dosing.

Expanding upon the above, release of a drug can be modified such as to achieve other than immediate release of the drug to thereby provide greater control of drug administration. For example, the formulation containing the drug can include, without limitation, an enteric coating and/or various components to obtain enteric release and/or controlled release of the drug. For example, enteric coatings prevent release of the drug in an acid medium but permit release of the drug in neutral or alkaline environments.

Controlled release formulations may have various release patterns. The release patterns can comprise one or more of prolonged release, sustained release, delayed release, and extended release formulations. Controlled release formulations are typically used to improve effects of drug administration by optimizing the kinetics of delivery, thereby increasing bioavailability, convenience, and patient compliance, as well as minimizing side effects associated with inappropriate immediate release rates such as a high initial release rate and, if undesired, uneven blood or tissue levels.

The term bioavailability is used to describe the degree to which a drug becomes available at the site(s) of action after administration. The degree and timing in which an agent, such as a drug, becomes available to the target site(s) after administration is determined by many factors, including the dosage form and various properties, e.g., dissolution rate of the drug.

Exemplary controlled release formulations include, without limitation, specially coated pellets, microparticles, implants, tablets, minitabs, orally disintergrating tablets, liquids, and capsules in which a controlled release of a drug is brought about. For example, controlled release can be through selective breakdown of the coating of the preparation, through release through the coating, through compounding with a matrix to affect the release of a drug, or through a combination of these techniques. Some controlled release formulations provide for pulsatile release of a single dose of an active compound at predetermined periods after administration. Also, for example, the controlled release formulation can include an amount of enteric and sustained release polymer from various polymeric materials which can be used to achieve a type of pattern of release needed to result in the desired plasma concentration profile, for example, so as to obtain a delivery over 24 hours.

Also, for example, the dosage form can be one unit or a plurality of units with the units being administered at the same time to achieve once-a-day dosing. For example, administration at the same time includes simultaneously administering multiple dosages or administrating multiple dosages within a reasonable period of time that permits a patient to comfortably swallow the units and/or administering multiple dosages within a time period that permits obtaining the effects of a single daily dose. The units can comprise any combination of strengths to achieve the desired daily dose as a once-a-day formulation. For example, to achieve a daily dose of 350 mg of clozapine, one unit of 350 mg can be administered, or a plurality of dosages achieving 350 mg of clozapine a day, such as units of 37.5 mg, 50 mg, 75 mg, 100 mg, 150 mg, 200 mg or 300 mg can be administered in any combination to provide the one-a-day dosing.

The once daily dosage can be administered any time during the day, and is preferably administered at the same time or about the same time each day. The dosing can be in the morning before or after breakfast or when the patient awakes. Moreover, the once daily dosage is preferably administered in the evening before bedtime, and even more preferably at bedtime. In this manner, side effects can be minimized. It is planned to have the time to peak clozapine plasma level to coincide with night time and sleeping period where due to clozapine the high risk of orthostatic dizziness will occur and where there is increased potential of sedation and somnolence For example, dizziness will not be noticed if the clozapine is administered, and the patient is asleep shortly after the administration. Accordingly, the administration at bedtime can be planned for the patient being asleep at the time of expected onset of potential side effects, such as dizziness.

Polymeric material of the controlled release formulation can comprise various polymeric materials, including, for example, cellulose acetate phthalate, cellulose acetate, cellulose acetate butyrate, cellulose acetate propionate, ethyl cellulose hydroxypropyl methylcellulose phthalate, polyvinyl acetate phthalate, hydroxy ethyl methyl cellulose. hydroxypropyl methylcellulose acetate succinate, microcrystalline cellulose, cellulose acetate trimellitate, hydroxypropyl methylcellulose succinate, cellulose acetate succinate, cellulose acetate hexahydrophthalate, cellulose propionate phthalate, copolymer of methylmethacrylic acid and methyl methacrylate, copolymer of methyl acrylate, methylmethacrylate and methacrylic acid, copolymer of methylvinyl ether and maleic anhydride, ethyl methyacrylate-methylmethacrylate-chlorotrimethylammonium ethyl acrylate copolymer, zein, shellac, copal collophorium, carboxymethyl ethylcellulose, co-polymerized methacrylic acid/methacrylic acid methyl esters polyvinyl acetate, fatty acid, fatty acid ester, alkyl alcohol, poly(ethylene oxide) and combinations thereof.

The controlled or modified release of clozapine desirably results in a mean plasma concentration profile in human psychotic patients, which over the course of 24 hours, given once daily dose (QD), will give an extent of absorption as measured by AUC equivalent to the same dose given twice daily (BID) but with a reduced Cₘₐₓ, such as at a dosage of 200 mg of clozapine, a Cₘₐₓ of 200 ng/mL to 500 ng/mL. The reduced Cₘₐₓ can provide for reduction of adverse effects associated with dose titration including orthostatic hypotension and seizures, and other adverse cardiovascular side effects, and may have an anti-cholinergic affect.

Moreover, while Cₘₐₓ is reduced in the once-a-day controlled release dose compared to the twice daily immediate release dose, Cₘᵢₙ in addition to AUC are desired to be similar to the twice daily dose but can be 60 percent lower in the once-a-day controlled release dose as compared to the twice daily dose. Thus controlled release (CR)Cₘₘ can be similar to immediate release (IR) BID Cₘᵢₙ or at least 60 percent of Cₘᵢₙ at steady state, and AUC can be similar to immediate release AUC or is at least 60 percent of BID at the same daily dose, while Cₘₐₓ is less than the equivalent BID Cₘₐₓ for immediate release clozapine.

Expanding upon the above, in a 12.5 mg immediate release clozapine BID, the resultant Cₘₐₓ is typically 15 to about 50 ng/mL. In a 25 mg once daily controlled release which can be a single component controlled release dose or multicomponent dose with combinations of immediate release and controlled release components, the Cₘₐₓ can be 5 to 40 ng/mL.

For 100 mg immediate release clozapine given BID corresponding to 200 daily dose in steady state the resultant Cₘₐₓ is 300 to 700 ng/mL. If this is given as 200 mg controlled release QD dose to steady state, then the resultant Cₘₐₓ would be in the range 200 to 500 ng/mL

The clozapine formulation can be in any form that provides for oral administration in controlled release form, and can include, without limitation, administration as tablet, capsule or orally dissolvable tablet (ODT), powders, solutions, suspensions, soft gel capsules, hard gel capsules, or chewable tablet.

The controlled release formulation can include an immediate release portion, such as wherein a percentage of 10 to 50% of the active ingredient is present as immediate release component. For example, the controlled release formulation can include immediate release component of less than or equal to 25 mg clozapine in addition to a controlled release component. Thus, for example, various components can be included in the controlled release formulation as long as the formulation is a controlled release formulation, and provides the advantageous rapid titration and/or titration at higher initial titration dose. Thus, the once-a-day controlled release clozapine can comprise a dosage form including an immediate release component as long as there are achieved the once-a-day controlled release.

For example, suitable modified release systems include controlled release and delayed coatings, e.g., on beads or tablets or matrix type release tablet with a polymer swell and erosion and osmotic systems, etc.

Modified release beads can be prepared by coating drug-containing cores with a water-insoluble polymer, or a combination of water-insoluble polymers, or a combination of water-insoluble and water-soluble polymers. The resultant beads can then be placed in a capsule or compressed into a tablet.

Polymers can be pH-dependent which have dissolution profiles which vary according to changes in the in-vitro dissolution media, or due to passage of the dosage form through the gastrointestinal tract. The polymers can also be pH-independent which are substantially unaffected by pH in vitro or in-vivo.

The pharmaceutical composition can comprise a formulation suitable for a once-a-day administration of clozapine. The formulations can include, without limitation, specially coated pellets, microparticles, implants, tablets, minitabs, and capsules. Such once-a-day formulations can comprise, for example, controlled release solid, clozapine bearing particulates, at least a portion of which releases clozapine in the lower gastrointestinal (GI) tract, such that once-a-day administration of said pharmaceutical composition provides steady state blood levels of clozapine that are comparable to steady state blood levels of clozapine that achieved with twice daily administration of about 100 to 1200 mg immediate release clozapine tablets per day daily dose. The particulates can comprise, for example, at least one polymer selected from enteric polymers, release controlling polymers, or combinations thereof.

Thus, formulations can be used that administer clozapine once daily (QD). There apparently is currently no clozapine QD being marketed in the US or Europe. In the US, clozapine tablets are typically taken twice daily. QD dosing of the clozapine has advantages associated with once daily administration. Better patient compliance and less healthcare professional supervision associated would be needed with one dose rather two doses per day for a patient population who have a higher propensity to not want to take their medication. Clozapine in common with another related second generation atypical antipsychotic, quetiapine, has a distinct advantage over other antipsychotics in that clozapine does not elicit motor side effects such as muscle stiffness or tardive dyskinesia. Motor side effect is due to the extent of clozapine blocking dopamine D2 receptors in the brain along with how quickly it dissociates from the D2 receptor. Clozapine optimally blocks the receptors and quickly desorbs from it hence minimizing motor effect compared to many other antipsychotics. But by quickly dissociating from D2 receptors, clozapine loses effectiveness and it is important that the dosage regime be adhered to or the patient can decompensate. As clozapine administered in a controlled release formulation will affect better patient compliance, and therefore would also be of significant patient benefit.

The patient can be a mammal, and preferably is a human.

The clozapine controlled release formulation can be made in any manner that permits the controlled release of the clozapine, and preferably a controlled release of the clozapine for once-a-day administration. For example, the formulation can comprise formulations including, without limitation, specially coated pellets, microparticles, implants, tablets, minitabs, and capsules that provide for controlled release of clozapine, preferably in a once-a-day formulation. As discussed above, the formulations can include various components, and can include, for example, two or more components. For example, the formulation can include an immediate release component and a controlled release component, or two controlled release components. One of the controlled release components, such as the second controlled release component can include, for example, a lag or delay. The term controlled release, CR, is used here as it is a common term used in the industry and the term the FDA uses. Other interchangeable terms with controlled are modified, prolonged and extended release. CR refers to a continuous or sustained release (SR) rather than immediate release, IR. The term delayed release, DR, includes any type delay or lag when releasing the active ingredient, and can include an enteric coated formulation which is kept intact in the stomach, termed a lag, and released in the gut. CR also refers to the totality of components in the formulation, such as a capsule, with at least one component being a CR component. Thus, as noted above, the formulation, such as a capsule, can include an immediate release component; however, the formulation by having at least one controlled release component will be considered to be a controlled release formulation.

### EXAMPLES

The following examples are for illustrative purposes only and are not intended to limit the scope of the present invention. Unless otherwise stated, the units are percent by weight.

Traditional in vivo methods using preclinical animal models, such as rat, dog or monkey, are not always sufficient to cope with a demand for development and testing of pharmaceutical formulations. In vitro and in silico Absorption, Distribution, Metabolism and Excretion (ADME) screening have been used for a number of years to move quicker into in vivo studies. In silico modeling for absorption prediction of clozapine was carried out. Particularly, absorption data from previous in vivo clozapine (FazaClo® immediate release BID) PK studies were reviewed and used to develop mathematical models. The models can be used to describe predications of once daily controlled release absorption in terms of absorption constant. Plasma concentrations achieved can be controlled by the rate of drug absorption.

There are limitations to a steady state model based on single dose data such as:

Underlying assumptions that elimination half-life is the same after multiple dosing relative to that after single-dose administration;

Saturable gut wall metabolic processes indicating concentration-dependent pharmacokinetics;

Potential for a food effect on multiple dosing;

Effect of circadian variation in absorption or metabolism; and

GI regional absorption differences resulting in reduced or increased bioavailability when drug is released over prolonged interval or intentional delay in release.

The modelling can be completed using WinNonlin software (Pharsight Corporation Version 4.0.1). Various mathematical models can be evaluated with first order absorption and eliminations with 1 or 2 compartments with and without associated lag time.

Assuming the PK parameter absorption rate constant, K01, is 1.504. Adjustment of K01, results in a reduction in the peak concentration (Cₘₐₓ) and time to reach peak.

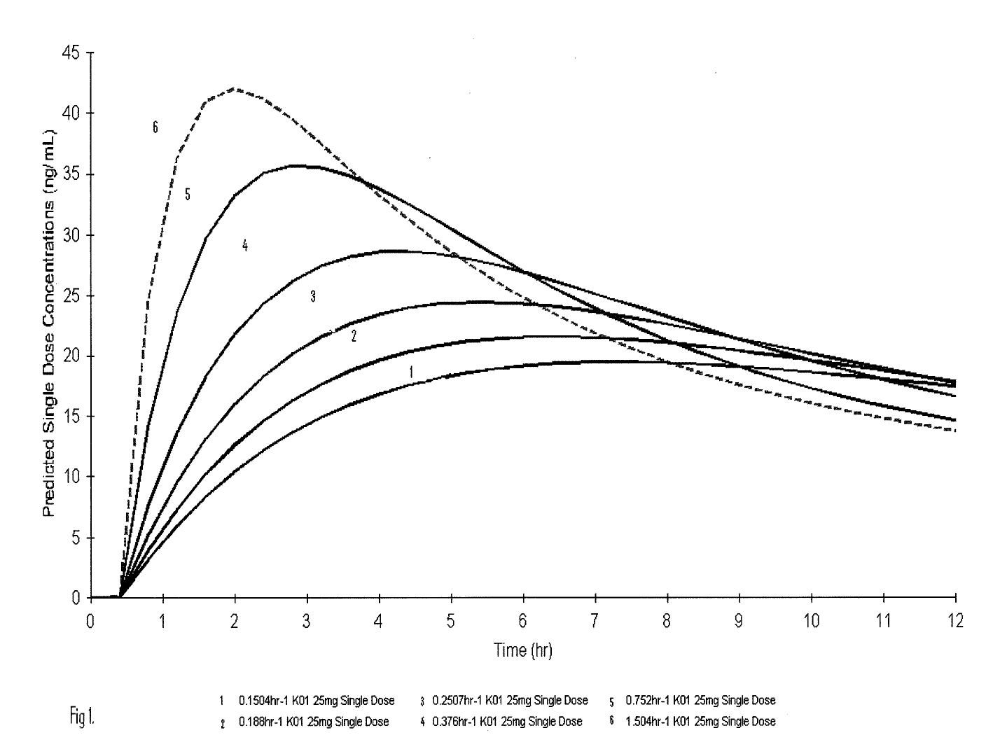

In the Example illustrated in FIG. 1, an in silico example for a 25 mg clozapine single dose, the reference K01 value is modified by factors of 1/10, 1/8, 1/6, ¼, and ½ so that the Cₘₐₓ can be evaluated. Line or curve 6 represents the reference immediate release (IR) dose. This indicates that the Cₘₐₓ and time to reach Cₘₐₓ can be shifted considerably by altering the rate of input.

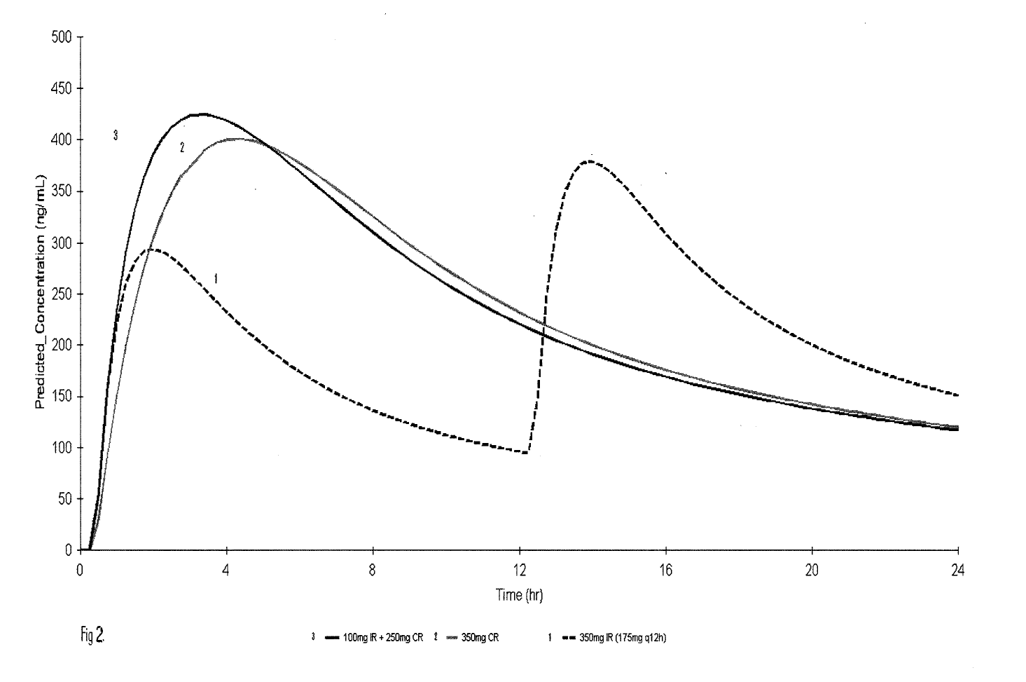

In the Example illustrated in FIG. 2, line 1 represents a 175 mg IR dose given at 12 h intervals, i.e., 175 mg BID. There are two controlled release (CR) simulations wherein the reference K01 value is modified by a factor of ¼. Line 3 is a dual component CR, i.e., it has an IR component of 100 mg and a CR component of 250 mg. The line 2 represents a one component CR (in this case there is no IR) only 350 mg CR.

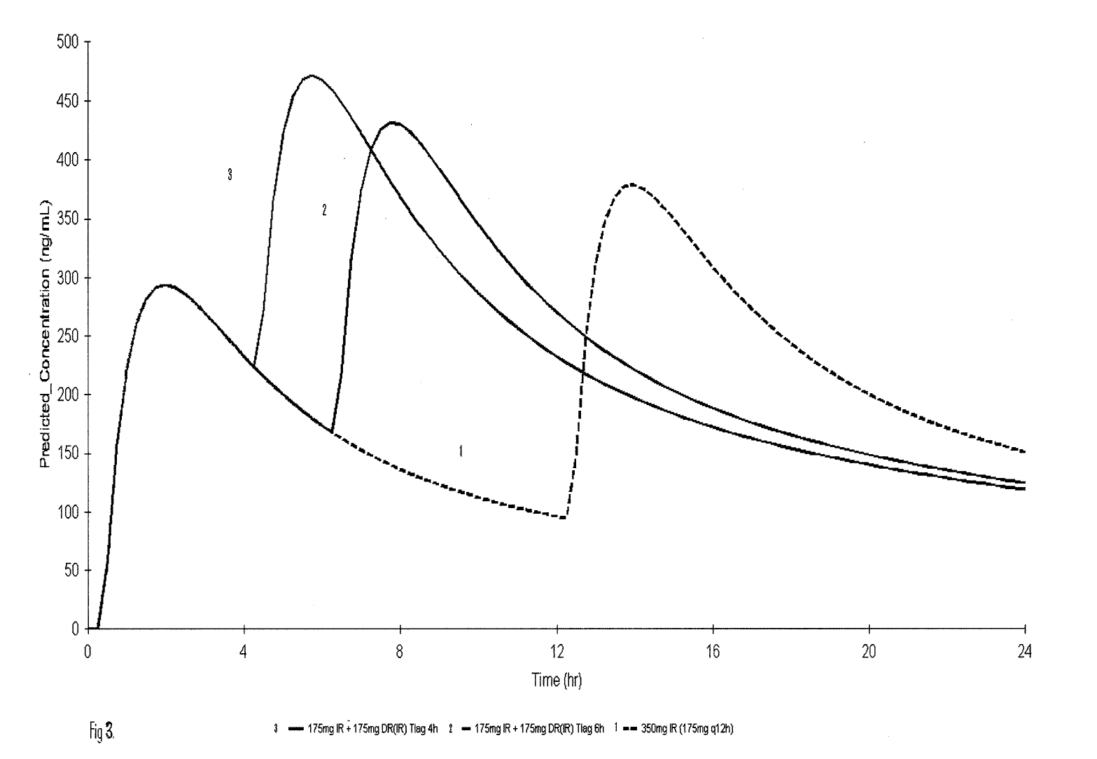

In the Example illustrated in FIG. 3, the CR component comprises delayed release components. Again, there is reference IR BID given for comparison along with two simulations including combinations of IR and delayed release. There is no sustained release type CR component. Line 1 is the BID; 175 mg IR dosed at 12 h intervals.

These are simulations for including an immediate release component in the delayed release formulation. One simulation has a delayed release component at 4 hours and the other simulation has a delayed release component at 6 hours. In these cases, the Cₘₐₓ is not reduced due to lack of sustained release for the second pulse. The first pulse in all is a common IR component and will therefore have the same Cₘₐₓ for the first pulse.

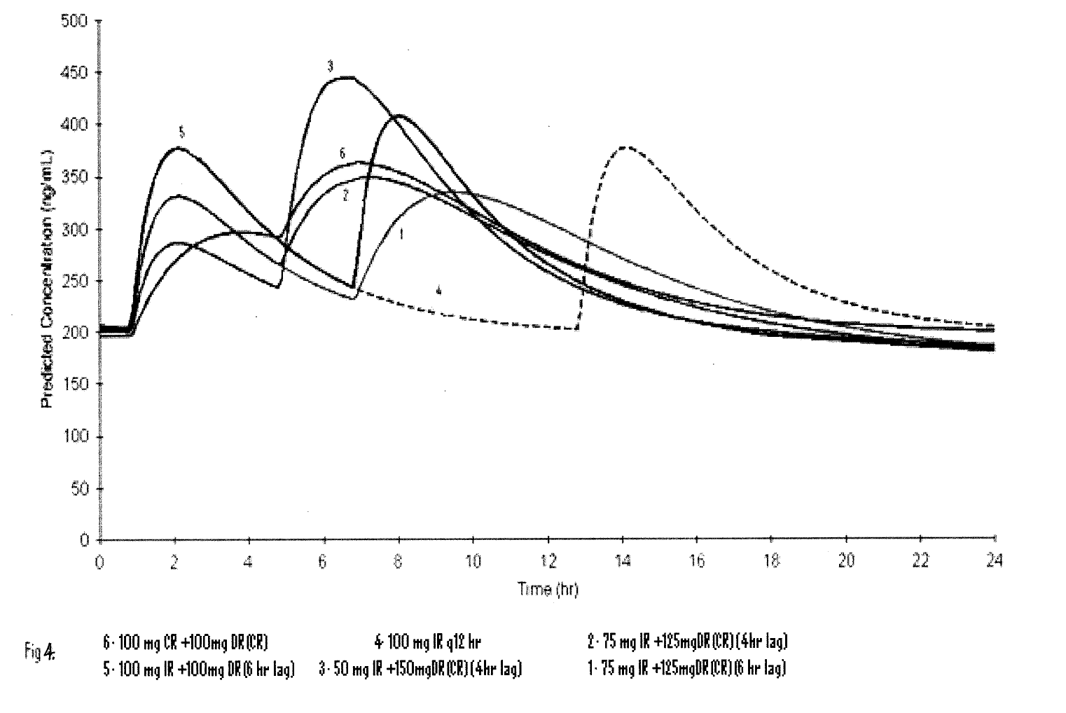

In the Example illustrated in FIG. 4, there are five CR simulations this time based on steady state data. There is also the BID reference. Thus, in the following example CR simulations are based on the modeling given in the previous examples. Each is made up of two components, the initial is an immediate release or controlled release component, and the second component is a controlled release component. Appropriate lags of 4 and 6 hours were also added to the second component. Formulations were simulated based on K01 by 5.

| | AUCₜₐᵤ | Cₘₐₓ | Cₘᵢₙ |
|---|---|---|---|
| | (ng/mL · hr) | (ng/mL) | (ng/mL) |
| 100 mg IR + 100 mg Delayed IR | 6275.2 | 407.2 | 180.1 |
| (6 hr lag) | | | |
| 75 mg IR + 125 mg Delayed CR | 6229.8 | 349.2 | 182.0 |
| (4 hr lag) | | | |
| 75 mg IR + 125 mg Delayed CR | 6220.6 | 334.6 | 184.5 |
| (6 hr lag) | | | |
| 50 mg IR + 150 mg Delayed CR | 6262.4 | 446.0 | 185.3 |
| (4 hr lag) | | | |
| 100 mg CR + 100 mg CR (K01) | 6300.6 | 362.5 | 199.5 |
| (4 hr lag) | | | |
| 100 mg IR BID (q12 hr) | 6314.0 | 376.7 | 203.1 |

The examples show that the bioavailability for the simulations is similar to the reference and that the Cₘₐₓ can be modified and can be reduced. There is no loss in bioavailability built into model as it is not known and not quantifiable and the impact of circadian variation is unknown.

All documents cited herein are incorporated by reference herein in their entireties as if their disclosures are set forth in full.

Although the present invention has been described in considerable detail with regard to certain versions thereof, other versions are possible, and alterations, permutations, and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

What is claimed is:

1. A method for titrating clozapine in treatment of a psychotic disorder in a human patient in need of treatment with clozapine, comprising administering once-a-day controlled release clozapine to the human patient and titrating to a therapeutic dose within a period of less than one week.

2. The method according to claim 1 wherein the once-a-day controlled release clozapine is administered at an initial dose of 25 mg.

3. The method according to claim 1 wherein the therapeutic dose is a daily dose of at least 300 mg of clozapine.

4. The method according to claim 1 wherein the therapeutic dose is a daily dose of at least 350 mg of clozapine.

5. The method according to claim 1 wherein the therapeutic dose is a daily dose of at least 400 mg of clozapine.

6. The method according to claim 1 wherein the therapeutic dose is a daily dose of at least 450 mg of clozapine.

7. A method for titrating clozapine in treatment of a psychotic disorder in a human patient in need of treatment with clozapine, comprising administering once-a-day controlled release clozapine to the human patient at an initial dose of at least 37.5 mg of clozapine and titrating to a therapeutic dose within a period of less than ten days.

8. The method according to claim 7 wherein the administering comprises obtaining a therapeutic dose in one week.

9. The method according to claim 7 wherein the administering comprises obtaining a therapeutic dose in less than one week.

10. The method according to claim 7 wherein the administering comprises obtaining a therapeutic dose in four days.

11. The method according to claim 7 wherein the administering comprises obtaining a therapeutic dose in three days.

12. The method according to claim 7 wherein the administering comprises obtaining a therapeutic dose in two days.

13. The method according to claim 7 wherein the once-a-day controlled release clozapine comprises a dosage form including 37.5 mg of clozapine in a single unit.

14. The method according to claim 7 wherein the initial dose comprises 50 mg of clozapine.

15. The method according to claim 7 wherein the initial dose comprises 100 mg of clozapine.

16. The method according to claim 7 wherein the titrating comprises increasing each once-a-day dose following the initial dose by at least 50 mg of clozapine.

17. The method according to claim 14 wherein the titrating comprises increasing each once-a-day dose following the initial dose by at least 50 mg of clozapine.

18. The method according to claim 7 wherein the titrating comprises increasing each once-a-day dose following the initial dose by at least 100 mg of clozapine.

19. The method according to claim 7 wherein the titrating comprises increasing each once-a-day dose following the initial dose by at least 200 mg of clozapine.

20. The method according to claim 7 wherein, when the once-a-day controlled release clozapine comprises 200 mg of clozapine, there is obtained a steady state blood Cₘₐₓ level of clozapine in the range of 200 ng/mL to about 500 ng/mL.

21. The method according to claim 20 wherein the daily dose of 200 mg of clozapine obtains steady state blood Cₘᵢₙ of clozapine of 50 to 350 ng/mL.

22. The method according to claim 20 wherein the AUC is 4000 to 10000 ng/mL*hr.

23. The method according to claim 7 wherein the daily dose is administered in the evening before bedtime.

24. The method according to claim 7 wherein the daily dose is administered at bedtime.

25. The method according to claim 7 wherein the daily dose is administered at the morning before breakfast.

26. The method according to claim 7 wherein the daily dose is administered at the morning after breakfast

27. The method according to claim 7 wherein the daily dose is administered at a specific time throughout the day.

28. The method according to claim 7 wherein an improvement in schizophrenia occurs as measured by Positive and Negative Syndrome Scale (PANSS).

29. The method according to claim 7 wherein there is an improvement in patient mood, a slower onset of sedation, and/or a lessening of sedation.

30. The method according to claim 7 wherein the administering of clozapine is performed to minimize central nervous system complaints; autonomic nervous system complaints; cardiovascular findings; gastrointestinal complaints; and/or miscellaneous complaints.

31. The method according to claim 7 wherein the administering of clozapine is performed to minimize agranulocytosis, seizures, myocarditis, electrocardiogram (ECG) repolarization changes, hyperglycemia and diabetes mellitus; other adverse cardiovascular and respiratory events and/or anti-cholinergic affect.

32. The method according to claim 7 wherein the administering of clozapine is performed to minimize orthostatic hypotension, respiratory and/or cardiac arrest, and cardiomyopathy.

33. The method according to claim 7 wherein the therapeutic dose is a daily dose of at least 100 mg of clozapine.

34. The method according to claim 7 wherein the therapeutic dose is a daily dose of at least 300 mg of clozapine.
